(19) United States
(12) Patent Application Publication
(10) Pub. No.: US 2009/0269415 A1
(43) Pub. Date: Oct. 29, 2009

(54) NOVEL THERAPEUTIC AGENT DERIVED FROM MARINE ORGANISM

(75) Inventors: Foo Wah Foong, Kyoto (JP); Masato Kuwabara, Kanagawa (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: IMEX JAPAN CO., LTD., Kyoto-shi, Kyoto (JP)

(21) Appl. No.: 12/065,414

(22) PCT Filed: Aug. 31, 2006
(86) PCT No.: PCT/JP2006/317254

§ 371 Date: Oct. 29, 2008

(30) Foreign Application Priority Data
Aug. 31, 2005 (JP) 2005-252684

(51) Int. Cl. A61K 35/56; A61P 25/00; A61P 3/00; A61P 29/00; A61P 37/04

(52) U.S. Cl. 424/520

(57) ABSTRACT

There is provided a nerve repairing agent, anti-ulcer agent or the like comprising an extract from a marine organism selected from organisms belonging to the class Holothuroidea, the phylum Porifera and the order Actiniaria or a material removed from a viscus of the organism. The extract has the following properties (1) to (4): (1) it is a almost colorless, transparent and viscous liquid having a slight fishy smell, or a almost white powder having a slight fishy smell which, when dissolved in water, is almost colorless and viscous; (2) a solution prepared by dissolving the liquid or powder in water shows the maximum absorbance at a wavelength of 264±10 nm; (3) a solution prepared by dissolving the liquid or powder in water shows an acidic pH value; and (4) it has at least one effect selected from a cranial nerve repairing effect, a nerve-growing effect, an insulin-releasing effect, an immunopotentiating effect, a noxious pain preventive effect, a neurogenic pain preventive effect, a diabetic sensory neuropathy ameliorating effect and an ulcer preventive effect.

### TECHNICAL FIELDS

The present invention relates a novel therapeutic agent derived from marine organism; especially which indicates neuronal repairs, anti-diabetic effect, immunoenhancement effect, analgesic action.

### BACKGROUND TECHNOLOGY

Sea cucumber (Holothuroidea) is one type of Echinodermata animals characterized with tissues that can alter its body hardness or muscle tone. The habitat of said sea cucumbers extends from shallow to deep sea waters, and these invertebrates are found in the seabed of tropical and subtropical regions. Sea cucumbers have long been consumed in Japan, and have been used as a nourishing item along the coasts of Asia. Sea cucumbers contain mucopolysaccharides such as chondroitin sulphate, etc., proteins such as collagens, and abundant amounts of minerals such as sodium and calcium, etc.

In recent years, studies of sea cucumber extracts have been investigated. For instance, tokukaihei 06-287111 has indicated that sea cucumber extracts to increase and promote hair growth while tokukaihei 2002-519384 has evidenced the relieving effects of sea cucumber tissue fractions on inflammatory symptoms and arthritis.

Furthermore, research studies have provided evidence to substantiate the present inventors have discovered that consumption of a sea cucumber extract may yield enhancement of the immunosystem to improve immunodeficiency-related symptom such as allergies, atopy etc., menopausal symptoms, menstruation disorders, to prevent and improve tumorous or malignant diseases that may be related with metastasis such as that found in breast cancer, to buffer the various signs and symptoms related with impairments of the liver and gastrointestinal systems, etc. such as diarrhea, may yield relieving effects on various symptoms related with the cardiovascular system such as hypertension or diabetes etc., elicit anti-inflammatory and analgesic effects, metastasis, and promote metabolic activity.

However, since the underlying mechanisms of action of these effects remain unknown, the active substance has to be identified and specified in detail.

[patent reference 1] Tokukaihei 06-287111

[patent reference 2] Tokuhyou 2002-519384

### DISCLOSURE OF THE INVENTION

### Problem to be Solved by the Invention

The present invention was based on said background technology with the objective to isolate the novel and highly active marine extract with least debris to provide various therapeutic agents derived from relevant marine organisms.

Under said background technology, the present inventors endeavored with much precise and strenuous effort to complete the present invention; from isolation of a certain specific fractionate to demonstration of relevant therapeutic effects thereof.

### Means for Solving the Problem

Said invented extract is characterized by that the extract is isolated from marine organisms, from any one species of sea cucumbers, sea cotton or sea anemones, or from said marine organisms where the internal organs/tissues are removed, which indicates the following characteristics: (1) an almost colorless liquid with a slightly fishy smell and stickiness or an almost white powder with fishy smell that dissolved in water to yield an almost colorless sticky liquid; (2) said liquid extract or water solution of said powder manifest peak absorbance at 264±10 nm; (3) said liquid extract or water solution of said powder indicate acidity pH; and (4) said liquid extract or said powder indicates at least one of the following features: brain neuronal repairs, nerve growth effects, endogenous insulin release, immunoenhancement effect, antinociceptive action, suppressive effect of neuralgia, improvement of diabetic peripheral neuropathy, and suppressive effect on ulceration at least.

Said extract is obtained by a step of boiling liquid which sea cucumbers without the skin and internal organs was liquefied under the sunlight, or a step of extracting from sea cotton or sea anemones with boiled water, a step of desalination of the liquid after boiling or the extract, a step of treating the desalinated liquid with active carbon, a step of removing of lipids and fats from the liquid treated with active carbon and a step of passing the defatted liquid through-cationic and anionic ion-exchange resins.

Said extract when administered to humans, animals and birds etc. improves neural abnormalities, promotes endogenous insulin release, enhances immunity, antinociceptive action, a suppresses neuralgia, improves diabetes-related neuropathy, and buffers ulceration. In other words, said invented extract may be used as a nerve repair agent, an antidiabetic, an immunoenhancement agent, an analgesic, an anti-ulceration or anti-inflammatory agent.

### EFFECTS OF THE INVENTION

According to the present invention, a novel and highly safe therapeutic agent derived from natural organism can be provided.

### BRIEF DESCRIPTIONS OF DRAWINGS

FIG. 1 The flow-chart indicating the manufacturing process of said invented extract (IJ-337)

FIG. 2 Absorbance spectra of said invented extract (liquid)

FIG. 3 Absorbance spectra of said invented extract (powder), when dissolved in water

FIG. 4 BDNF activation of said invented extract (IJ-337)

FIG. 5 NGF activation of said invented extract (IJ-337)

FIG. 6 Endogenous insulin release effect of said invented extract (IJ-337)

FIG. 7 NK/NKT activity enhancement effect of said invented extract (IJ-337)

FIG. 8 Macrophage activity enhancement effect (with single-bolus administration; BCL method) of said invented extract (IJ-337)

FIG. 9 Relative Th1/Th2 cell activity enhancement effect of said invented extract (IJ-337)

FIG. 10 NK/NKT activity enhancement effect (single bolus administration with more detail monitoring; BCL method) of said invented extract (IJ-337)

FIG. 11 Suppressive effects of said invented extract (IJ-337) on SP-induced nociception

FIG. 12 Buffering action of said invented extract (IJ-337) on hyperalgesia induced by nerve damage; (A) rat right hindlimb with sham operation per se, and (B) ligation of sciatic nerve of rat left hindlimb

FIG. 13 Improvement of diabetes-induced neuropathy (the nociceptive formalin test) by said invented extract (IJ-337)

FIG. 14 Antiulcerative effects of said invented extract (IJ-337) on ulcerative substance-induced ulcerations (ulcerative area as the index)

FIG. 15 Antiulcerative effects of said invented extract (IJ-337) on ulcerative substance-induced ulcerations (mortality rate as the index)

### BEST MODE FOR CARRYING OUT THE INVENTION

The extract of the present invention, which is an isolate derived from marine organisms such as sea cucumbers, sea cotton or sea anemones etc., possesses various pharmacological actions. Sea cucumbers of the present invention mean sea organisms belonging to Holothuroidea de Blainville, for instance, sea cucumbers (Stichopus japonicus, etc.) inhabiting seabed of coastal and deep sea waters in temperate regions, and sea cucumbers (Stichopus badionate Selenka, etc.) harvested in coastal seas in South-east Asia inclusive of Malaysia and other tropical and subtropical regions are listed. Said invented extract can be isolated from raw materials of any kind of species. In addition, sea cotton and sea anemones found in coastal waters can serve as raw materials as well.

Sea cotton is an organism that belongs to the Profera. Sea cotton maybe divided into two categories: Calcarea having characteristic bone structures with calcium carbonate as the major component, and non-calcarea that contain no calcium carbonate. Said invention may be derived from either sea cotton type, the latter (non-calcarea), especially normal sea cotton type (Demonspongiea) where its skeletal structure is formed by sea cotton fibers is especially preferred. In addition, a majority of sea cotton types inhabit the sea, and fresh water sea cotton can be used so long the extract yields the desired effects in this invention.

Sea anemones (Anemonia sulcata) are Echinodermata that belong to Actiniaria, and are soft invertebrates. Said invented extract dose not confine to Actiniaria, and includes extracts isolated from organisms traditionally known as sea anemones, inclusive of morphologically similar boneless sea corals, Corallomprpharia, Zoantharia, Ceriantharia etc.

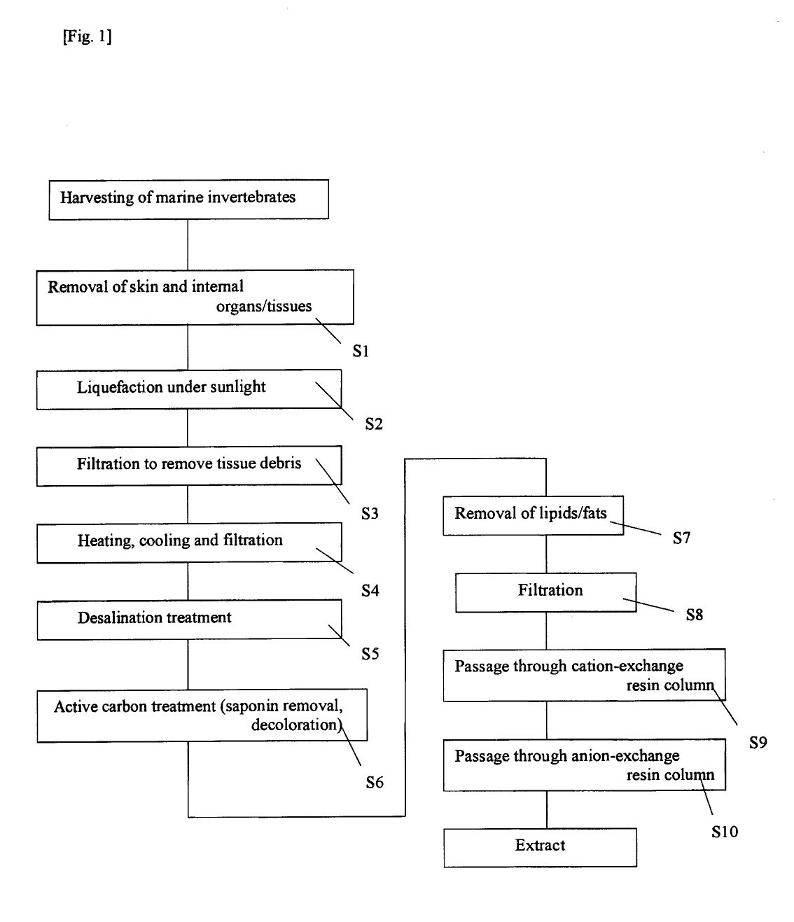

Said invented extract is for instance isolated by the methods illustrated in FIG. 1. The skin and internal organs/tissues are first surgically removed (S1). The tissues are then broken down by exposure to the sun and thereafter spontaneously melt to form a liquid mixture (S2). This phenomenon of liquefaction is a typical characteristic of the species called Stichopus badionate Selenka, etc. The liquid is filtered with for example glass wool and/or gauge cotton to remove the tissue debris (S3). Filtrate thus obtained is boiled and precipitates and debris produced are removed by paper filtration or decantation (S4). This filtration process is executed with the mixture in the heated state without cooling. The warm-to-hot filtrate is subjected to desalination with a well-known method without restricting to ion-exchange methods (S5). The desalted mixture is subjected to adsorption treatment by adding active carbon into the liquid (S6). This adsorption process is to remove saponin, stains and other substances (i.e. detoxification and decoloration procedures) from the liquid. So long it displays adsorption activity, active carbon used is therefore not a restricted and specifically specified substance. Next, lipids (fats) are selectively removed (S7). This fat-removal method for instance may be executed at 20 degree Centigrade to solidify fats through cooling, mixing said liquid with n-hexane or chloroform. Furthermore, an adsorption agent may also serve the purpose.

Thereafter, an ambient temperature higher than the fat-removal procedure is preferred; e.g. the process is performed under a room temperature of 27 degree Centigrade. Liquid in which lipids is removed is filtered through a filter (pore size: about 1 micro-meter) under about 6-bar pressure (S8). Filtrate is initially subjected to cationic ion-exchange (S9) followed by anionic ion-exchange (S10) to finally derive said invention extract. Filtration through the cationic and anionic ion-exchange, for instance, is achieved by passage of through columns filled with the relevant resins (may used columns previously filled with the relevant commercially available resins), where said invented extract is not absorbed by the resins and where said invention is the filtrate. Filtrate obtained in a step S8 indicates acidity pH. By passage through the cationic and anionic ion-exchange resins in the column under acidity pH, contaminants other than said invented extract are completely removed. Filtrate thus obtained (said invented extract) may further filtered through about 0.5 micro-meter (pore size) filter to remove anion-exchange resin and other insoluble impurities. Furthermore, to remove solving impurities yet in filtrate, step S6 to S10 may be repeated. In this case, the step for fat removal (S7) may be abbreviated. As such, it is possible to repeat the filtration process, back to the step S6 from the step S10, where the filtrate through anion-exchange resin is again subjected to adsorption by active carbon (S6), filtration (S8), filtration through cation-exchange resin (S9) and filtration through anion-exchange resin (S10). Note that the above procedure is merely an example; desalination method, fat-removal procedure and filter pore-size required for isolating said invented extract are not limited to the above specification and descriptions. In addition, the adsorption treatment, filtration process and desalination may not be a fixed and designated order as described, appropriate processing and procedural orders, or when required, complementary filtration processes may be added or altered. For example, the order of process of step S6 and process of step S7 may be reversed, where fat-removal may precede desalination prior to adsorption by active carbon.

The extract thus obtained is acidic, more concretely pH value is 2.5-3.5, colorless, and is a hydrophilic liquid with a slightly fishy odor and viscosity. Said invented extract indicates peak absorbance at 264±10 nm. Measurement of the particulate distribution shows the particulate size (laser diffraction analysis) to be about 0.5-2 micro-meter. The concrete chemical composition of said invented extract is unknown. Said extract is heat-stable; its biological activities are not affected after heating to 150 degree Centigrade.

Said invented extract is obtained in a liquid form under normal conditions, when extracted from the method described above. Said invented extract, for instance, can be made to yield a powder when subjected to lyophilization. The powder is white with tinges of yellow to light brownish appearance. In addition, solutions derived from this powder indicate acidity with slight viscosity, and display peak absorbance of 264±10 nm. The pH value obviously varies with the concentration, and there are cases where the peak may shift slightly from said peak absorbance wavelength.

The harvest of said invented extract from sea cucumber has been described in the above description. Said invented extract can also be extracted from invertebrates similar to sea cucumbers, such as sea cotton and sea anemones and the like, although these species do not liquefy under sunlight exposure. Furthermore, there are cases where sea cucumbers other than Stichopus badionate Selenka may not liquefy under sunlight exposure. In these cases, intrinsically existing internals organs/tissues have to be removed and/or in the absence of any internal organs/tissues the marine organism is heated in water as a whole. The extract thus obtained is subjected to desalination, and treated in a similar manner as described for cases of the above-mentioned sea cucumbers rightfully as a liquid extract or as a lyophilized powder from the thus obtained extract.

This extract exhibits the following pharmacological activities. Said invented extract indicates brain neuronal repairing and nerve-growth effects. Or, said invented extract stimulates the releases of nerve growth factor (NGF) and brain-derived neurotrophic factor (BDNF). The NGF is a protein that promotes survival and/or growth differentiation of sensory ganglionic neurons and sympathetic ganglionic cells; at the least, it serves as a neuronal nutrient factor for cholinergic neurons in the basilar frontal brain. In diabetes-induced peripheral neuropathy and ulcerations/lesions, where neuronal damage is inflicted, NGF supply is disturbed to result in neurodegenerations. BDNF is a protein that displays neurotrophic effects on memory-associated cholinergic neurons of the cerebrobasal nucleus (sensory neurons of the peripheral nervous system); serves essentially for the survival of cerebral striatal neurons; acts as an indispensable factor for regeneration of development of synapses. Neurotrophic factors are also known to contain both NGF and BDNF. Neurotrophic factors are known to be useful for neurodegenerative disorders of various diseases similar to Alzheimer's and Parkinsonian diseases and/or degenerations and attenuated differentiation of neurons induced by external trauma or physical impact on the spinal cord and brain stroke (Appl Ann Neuroplogy, 10 (1981), 499).

The said invented extract promotes the releases of NGF and BDNF and enhances neurotrophic factors. As such, said invented extract may be applied as a therapeutic agent for the promotion of repairs of nerve injuries in central nervous system or peripheral nervous system; i.e. as a neuronal repairing/regenerating agent. Said nerve-repairing agent of the present invention therefore promotes repairs of neurons and reconstruction/restoration of synapse connections, so said invented extract may be administered for possible treatment of animals and humans suffering from diseases with typical features such as neurodegeneration and impairments of neuron-neuron connections/contacts, for instance, the aforesaid Alzheimer's disease, Parkisonism, Pick disease, striatal degeneration, Shy-Drager syndrome, Hallervordent-Spatz syndrome, progressive supranuclear palsy, olivopontocerebellar atrophy, Friedreich motor incoordination, capillary vasodilatory motor incoordination, amyotrophic lateral sclerosis, protopathic sclerosis, progressive muscle atrophy, progressive bulbar palsy, pseudobullar palsy, Werdnig-Hoffman disease, Kugelberg-Welander syndrome, multiple sclerosis, and venous cerebrospinal meningitis, etc.

In addition, the said invented extract induces release of endogenous insulin. The release of endogenous insulin is beneficial for the control of blood sugar levels. Insulin facilitates uptake of sugar, amino acids and potassium, promotion of glycogen syntheses and protein syntheses in muscles; facilitates uptake and use of sugar, promotion of syntheses and inhibition of degradation of fats, promotion of protein syntheses in fatty tissues, etc. When blood sugar levels increase after meals etc., insulin is secreted from the pancreas, high sugar levels can be restored to normal levels. Diabetes due to insulin deficiency or insulin insufficiency results in abnormal glucose metabolism, and excessive increase in the blood sugar contents to eventuate glucosuria, resulting in a persistent diseased state of chronic hyperglycemia. Hyperglycemia in turn may be complicated with neuronal damage, cataract, kidney impairments, retinopathy, atherosclerosis, arteriosclerosis, and diabetes-derived gangrene and other complications. Diabetes is categorized as type-I (insulin-dependent) and type-II (insulin-nondependent). Diabetes type-I is a pathological state where the pancreatic beta-cells does not secret insulin to eventuate hyperglycemia, while diabetes type-II is a state where either insulin is secreted in an inadequate amount (insulin deficiency) or the effect of insulin to suppressing blood sugar levels is weakened (insulin tolerance) to eventually yield hyperglycemia, may due be closely related with excessive calorie intake, imbalanced nutritional intake, inadequate exercise and stress other than genetics. Insulin secretion from the pancreas is necessary to afford proper control of blood sugar in type-II diabetes. Said invented extract promotes insulin release, and is therefore appropriate for use for insulin-nondependent diabetes type-II. As such, the said invented extract is potentially useful for the prevention and/or buffering diabetes regulation and complications.

The said invented extract indicates immunoenhancement effect. Long-term exposure to and/or contact with exogenous antigens in the environment induces abnormalities in the immune system of the living system. In addition, macrophage releases various active endogenous cell activities when abnormal cells such as tumors are detected. Especially, INF-gamma released by macrophage differentiates non-differentiated T-cells Th(T-helper)0 to Th1 cells. Furthermore IL-4 released by natural killer/natural killer T (NK/NKT) cells differentiates Th0 to Th2. IL-2 released from Th1 cells activates cytotoxic lymphocytes (CTL). IL-4, IL-5 and IL-6 activate released from Th2 activate B-cells to produce antibodies. In addition, macrophages not only activate other cells but also elicit per se activation. These activated cells These activated cells then suppress those exogenous antigens inside the body system. Furthermore, these activated cells collectively attack those abnormal tumor cells. As such, the activated T cells, B cells and macrophages build up and subsequently boast the immunodefense system to inhibit tumor cell proliferation and attenuate the damaging effects thereof. Additionally, enhancements of the other immunosystems are activated to elicit protective mechanisms against the various immunosystem-related diseases such as allergies and rheumatism. Said invented extract also enhances NK/NKT cell activity to eventually advocate that administration of said invented extract may serve as one of the hopeful immunotherapies in treating immunosystem-related diseases such as cancers, tumors, allergies, rheumatism, atopy, etc.

Said invented extract indicates suppressive effects on nociceptive responses and suppresses neuropathic pain. According to etiological factors, pains may be categorized into nociceptive responses which are pains occurred when healthy tissues are injured or the stimulus having the potential risk are added, neuropathic pains which are pathological pains induced abnormalities of the function of the peripheral and central nervous systems and idiopathic pains which are pains without appropriate anatomical explanation, or pains where physical lesions/injuries are not apparent.

Nociception is pain that is induced via a nociceptor, for typical instance pain induced by Substance P (SP). SP is a kind of neuron peptides. SP is extensively distributed in the peripheral and central nervous systems. Apart from acting as a transmitter in the primary sensory nerves, SP portrays various physiological effects such as vasodilatory effect, enhanced vasopermeability, smooth muscle contractility, neuronal excitability, salivary secretory effect, enhanced diuretic effect and immunoeffect. Of special note is the release of SP at the nerve terminal in the spinal dorsal horn evoked by a nociceptive impulse to subsequently transmit the nociceptive information in the form of chronic pain via the secondary neurons. SP released at the nerve terminals has long known to thereafter induce an inflammatory response via nociceptors. As such, SP is involved in various inflammation-related pathologies and diseases; e.g. chronic inflammatory disease such as common pain, headache (especially migraine), Alzheimer's disease, multiple sclerosis, cardiovascular variations/adjustments, chronic joint rheumatism; respiratory diseases such as asthma and/or allergic rhinitis; immunity-relevant inflammatory intestinal diseases such as ulcerative colitis and Crohn's disease; eyeball inflammation, eyeball injuries and proliferative vitreous retinopathy; irritable bowel syndrome, polyuria; psychogenic diseases; and vomiting. When SP was injected in the hindpaw planta of mice, nociceptive responses were reflected as scratching response. The present invention of said invented extract suppressed the scratching activity.

Neuropathic pain (neuronal impairment pain) is induced without the participation of nociceptors, although involves of primary injury/damage and the accompanying functional abnormalities are more likely to be the causes or the inducing factors as well as abnormal excitatory activities of both the peripheral and central nervous conduction pathways. The mechanisms of the peripheral neural mechanisms for such pain are: (1) morphological changes of neural fibres, (2) electrophysiological changes, (3) participation of ion channels, (4) changes to neuroactive substances, and (5) changes in the sympathetic nervous system; while those of the central nervous mechanisms are: (1) changes of sensitivity and pain transmission of the nerves, (2) structural changes, and (3) disturbance and/or hypofunctional events of the inhibitory nervous systems. For instance, by the report of Bennett and Xie (Pain 1988; 33(1):87-107), tying the sciatic nerve of the hindlimb gently reduced significantly the nociceptive threshold of the peripheral nociceptive stimulation as a result of neural damage and induced neuropathic hyperalgesia. Said invented extract suppresses the neuropathic hyperalgesia induced by the above-mentioned method.

Furthermore, said invented extract improves diabetes-derived sensory neuropathy. Diabetes is a state where the blood sugar of patients is constantly maintained at a high level as above mentioned. Long-term hyperglycemic conditions induce peripheral nerve disorder. Self-diagnosed symptoms include numbness, pinching pain, tingling sensation, burning sensation, piercing pain and abnormal pains (pain sensation against normal stimulation). Although the causes cannot be determined, thickening and occlusion of the basal membrane of neurotrophic blood vessels are noted. This vascular damage then reduces blood flow to nerves, causing eventual neural damage. Using the STP (streptozotocine)-induced mouse diabetes model, said invented extract buffers nociceptive responses when noxious mechanical stimulation is delivered to the animals.

When these effects are taken into consideration, said invented extract buffers and may treat nociceptive disorders and may serve as an analgesic agent including for suppressing (treating) diabetes-derived pains. Additionally, with the above-mentioned nerve-repairing and nerve growth effects, said invented extract may serve as a potential agent to buffer physical disabilities due to a cerebral stroke, diabetes-derived peripheral neuropathy and stress-induced anxiety, etc.

Said invented extract indicates anti-inflammatory and antiulcerative effects. Ulcerations is inflammatory external injuries which cause loss or damage on superficial tissues. Ulcerations are categorized into acute, subacute, chronic ulcers and recurrent ulcerations. The physiological mechanisms of membranous injuries in gastritis and digestive ulcerations is considered the unbalance between offensive factors such as acid production, pepsin and pylori-relations etc. and protective factors such as on-site mucus production, bicarbonates and blood flow, etc. Erosive gastritis is usually associated with severe diseases and/or oral drug intake. Stress, ethanol (excessive alcohol intake), bile, non-steroidal anti inflammatory agents such as indomethacin, steroids, antibiotics inflict the mucous wall of the stomach, allowing gastric juices, which normally are harmless, to erode the stomach wall. Although the mechanisms are unknown, said invented extract inhibits the progress and incidence of the aforesaid gastritis and ulcerations. The antiulcerative effects of said invented extract imply the inhibitory effects on symptoms and signs of gastric ulcers, duodenal ulcers, Crohn's disease, ulcerative colitis, irritable bowel syndrome and inflammatory enteropathy, etc.

Said invented extract may be applied to humans, dogs, cats, cattle, horses, rabbits and other animals, birds and any other species of living organisms as deemed fit. In addition, although said invented extract per se can be applied, it may mixed and/or combined with other pharmacologically non-active carriers to formulate and adjust into possible drug forms for administration purposes in experimental and clinical studies. In more concrete perspectives, in the forms of aerosols, capsules, emulsions, extracts, fluids, fludeextracts, granules, injections, infusions, liniments, ointments, plasters, powders, cataplasms, pills, suppositories, suspensions, syrups, tablets, tinctures, triturates, troches or other dosage forms. As carriers are exampled excipients such as starch, lactose etc., solvents such as water, ethanol, etc., and various water-soluble, oil-soluble ointment bases. Said invented extract may be mixed with the above-mentioned carriers, and other carriers including coating lubricants, coloring agents, flavoring, tinctorial agents, etc. to make into various dosage forms. Furthermore, said invented extract is the major active component with others dispensing as adjuvants, or it is also possible that said invented extract be used as a supplementary substance with others as the major active substances.

The administration route is not limited to oral use, intravenous, subcutaneous, intranasal, mucous injections and other administration routes may also be used. The administration dosage depends on the purpose and route; viz., 0.1-300 mg/kg body weight per administration from 1 to more administrations daily should be beneficial to patients or users.

### Example 1

As follows the invention is specifically explained by describing the examples. However, the invention is not restricted by the following examples.

(Manufacturing Extract)

Skin and international organs/tissues of sea cucumbers harvested along the coasts of peninsula Malaysia are removed. Sand and residual tissues were thoroughly washed with water before each organism was exposed to sunlight for 3 consecutive days to liquefy the invertebrates into a pulpy mixture and then filtered through cotton filters at room temperature (27 degree Centigrade) to completely remove tissue debris and residual matter. The crude filtrate was then heated to 100 degree Centigrade and again filtered with cotton filters to remove debris and tissue matter settled down at the bottom of containers. The filtrate was left to cool at room temperature (27 degree Centigrade). The filtrate was then passed through cationic resins and then anionic resins to remove salts. An appropriate amount of active carbon was added to the desalinated liquid to effect detoxification (desaponification, etc.) and decoloration by absorption. After cooling this mixture (about 20 degree Centigrade), the separated lipids (and other fats) were removed. The liquid is then kept at room temperature (27 degree Centigrade) and filtered through a filter (pore size: 1 micro-meter) under the pressure of about 6-bar. The filtrate is then passaged (filtered) through a cationic resin column (Lewatit MonoPlus S100; Sybron Chemicals) under about 6-bar pressure and then through an anionic resin column (Lewatit MonoPlus M500; Sybron Chemicals) under 6-bar pressure. The extract is treated with active carbon again before passaging the cationic (MonoPlus S100) and anionic (MonoPlus M500) columns again. The extract is filtered (pore size: 0.5 micro-meter) to isolate the active extract (to be named as IJ-337 thereafter). A portion of IJ-337 is lyophilize to powder.

(Physicochemical Properties of the Extract)

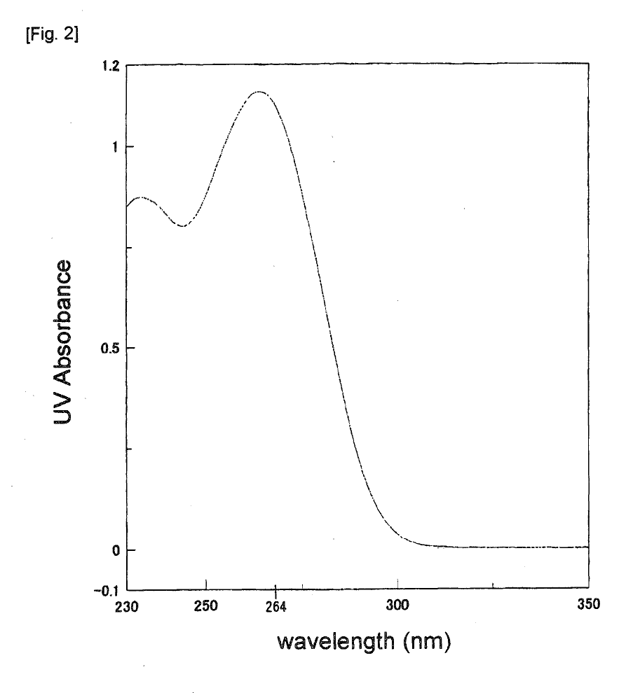

The IJ-337 thus obtained is a clear, colourless liquid with raw odor likely slight fishy at room temperature of about 27 degree Centigrade. The pH of IJ-337 is 2.9 and the specific gravity is 1.001. When IJ-337 is diluted 10 fold with distilled water, the peak absorbance is established at a wavelength of 264 nm using ultraviolet (UV) absorbance measurement as showed in FIG. 2. IJ-337(original solution) indicates absorbance or A₂₆₄=56.73 using spectrophotometry (UV/VIS spectrophotometer V-560; Nihon Bunkou Co. Ltd.). In addition, IJ-337 indicates a slimy touch (with viscosity).

A portion of IJ-337 is placed in a test-tube and heated to about 150 degree Centigrade, changes in color, appearance and odor are not apparent. Although data is not indicated herewith, the following pharmacological effects remain intact when tested with IJ-337 after heat treatment.

When IJ-337 is measured with a laser diffraction particulate distribution device, the particle size (by the laser diffraction method) ranges from about 0.5-2 micro meter. The mean molecular weight of the active extract contained in IJ-337 is 1000 or less than 1000 Dalton.

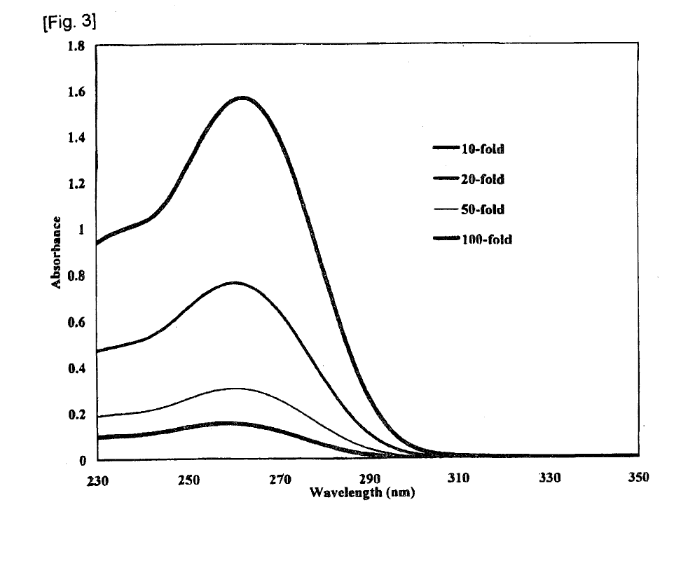

However, the lyophilized powder is white, odorless or has a slight fishy smell. An amount of 0.1 g dissolved in 100 ml indicates a pH value of 3.3. An amount of 3 mg dissolved in 1 ml and thereafter diluted 10, 20 and 50 folds for UV absorbance indicates the peak absorbance occurred at 270 nm using UV absorbance measurement as showed in FIG. 3. The solution of the extract is slightly viscous.

(Brain Neuronal Repairs and Nerve Growth Regeneration)

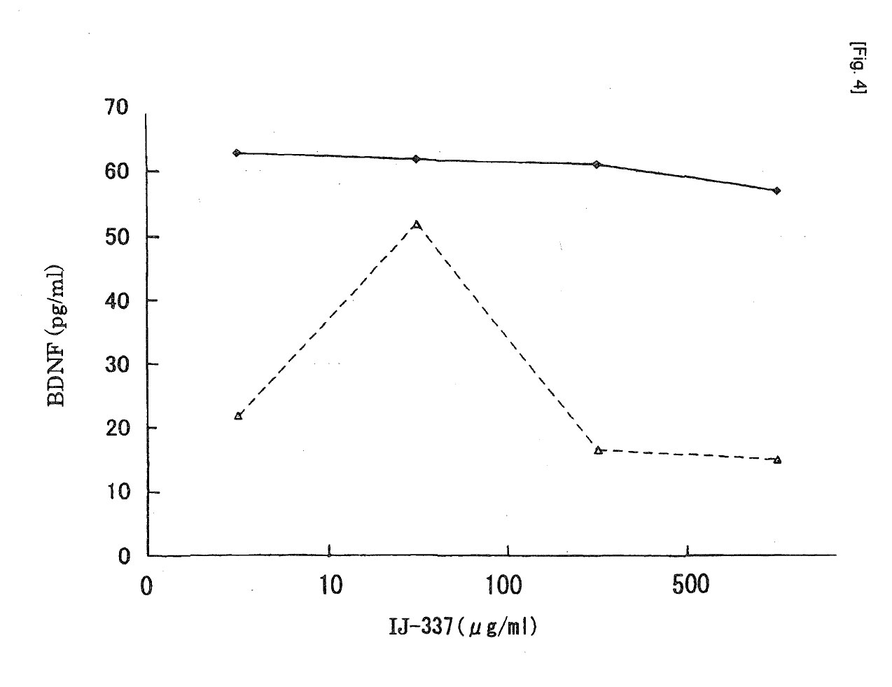

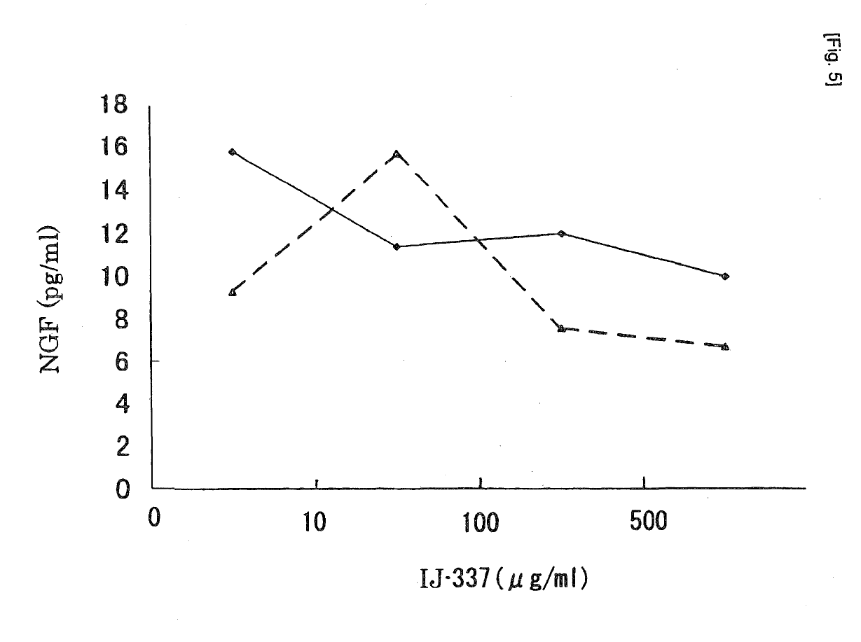

Whole brains of anesthetized 8-day-old ICR mice isolated was treated and cultured as described by Furukawa et al. (Biochem. Biophy. Res. Commun. 1986; 136: 57-63). Confluent astrocytes treated adequately described by Ohta et al. (Biochem. Biophy. Res. Commun. 2000; 272:18-22) were incubated with said invented extract (500-600 mg/mL) for 24 hr before measuring the levels of brain-derived neurotrophic factor (BDNF) and nerve growth factor (NGF). The results of IJ-337 effects were shown in FIG. 4 (BDNF) and FIG. 5(NGF). The results of the solubilized solution of IJ-337-treated cells (solid line) and the supernatant of the solubilized solution of IJ-337-treated cells (broken line) are indicated accordingly in the figures.

According to the results, BDNF and NGF levels were relatively higher than pretreatment, registering 2.5-fold (peak value: 53 pg/ml; FIG. 4) and 2.0-fold (peak value: 16 pg/ml; FIG. 5) increases respectively, thus suggesting that IJ-337 effects promoting brain neuronal repairs and nerve growth. Moreover, the most effective IJ-337 doses required to promote brain neuronal repairs and nerve growth were 10-100 micro gram/ml and the used amount indicating the maximum effect were 50-60 micro gram/ml, respectively.

(Anti-Diabetic Effects)

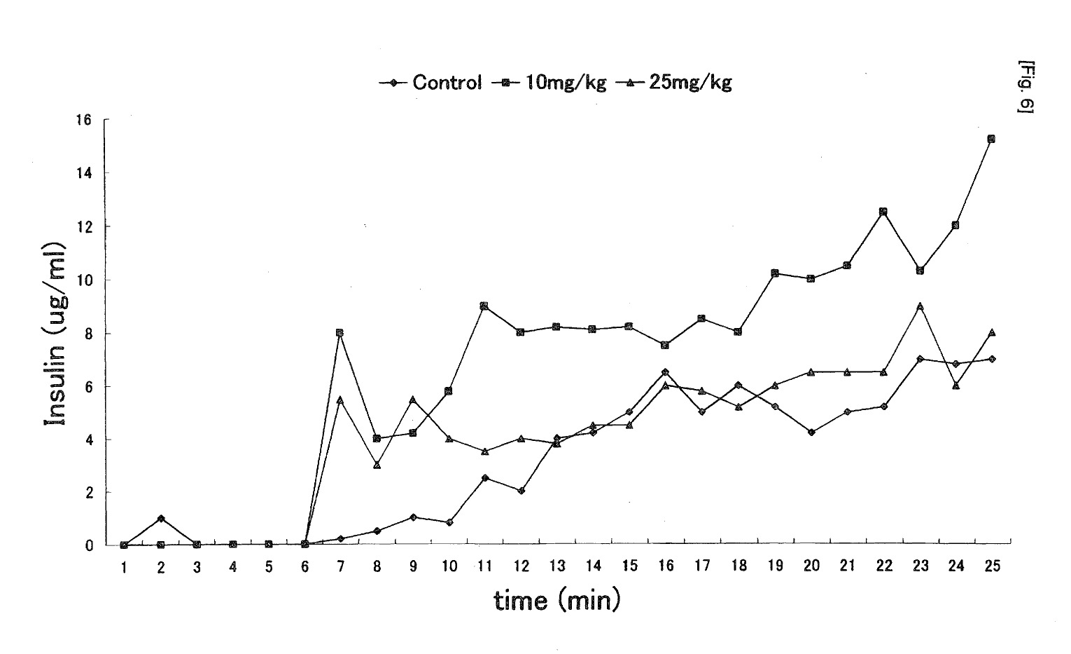

The endogenous insulin release in Wistar rats was investigated. Rats (body weight: 250-300 g) starved for 24 hr were anesthetized with pentobarbital (0.6 mg/kg) and the pancreas were exposed surgically as described by Grosky and Bennet (Diabetes 1960; 15: 910-13). A polyethylene tube was inserted in the insulin-secreting duct of the pancreas to collect samples of endogenously released insulin with time. IJ-337 was given to the rats at 10 and 25 mg/kg (p.o.) an hour before initiating measurements. As a control, endogenous insulin release with time was compared. The result was shown in FIG. 6. It was indicated that the release was abruptly increased 60 min after IJ-337 treatment, registering peak levels at 25 after initiation of experiment. Experiments were terminated 25 min after initiation of measurement. In oral administration of IJ-337 with a dose range of 1-25 mg/kg in rats, increases of the levels of endogenous insulin release indicated a dose-dependent fashion.

Although the release of endogenous insulin might have surpassed 25 min, the 25-min interval allocated for measurement was the limit under in situ experimental conditions. However, it is clear that IJ-337 induces and promotes release of endogenous insulin. These results imply that IJ-337 may serve a potentially useful agent for use in type-II diabetes and/or borderline patients with insulin deficiency.

(Immunoenhancement Effects)

The immunoenhancement effects of IJ-337 were next investigated. Two male Beagle dogs (10-11 kg) were accommodated and provided with feed pellets and drinking water ad libitum in a laboratory room maintained at a constant room temperature (25 degree Centigrade) and relative humidity of 50-60% for 1 week before experiment. IJ-337 (10 mg/kg) was administrated orally and blood samples were then taken with time. Sampling was done at 6 hr before, 6 and 48 hr after IJ-337 administration. Blood samples were collected via the jugular in heparin-treated test-tubes and assayed accordingly.

First, time-related changes of blood neutrophil activity were monitored. Blood samples were well agitated before being subjected to specific gravity centrifugation. The separated supernatant cells were collected as NK/NKT cells. Absorbed cells after separation were detached with trypsin, and were collected as macrophages. NK/NKT cell activities were measured using canine cell line of leukemia lymphocyte-1 (CL-1) as a stimulant and macrophage activities were measured using monocyte removal agent (Nihon Antibody Research Laboratories) as a stimulant, respectively. The immunity potential was measured using the BCL method.

Furthermore, the remaining heparin-treated blood samples were assayed for CD4/CD8 measurements (Hitachi: 5550; Hitachi Manuf. Co. Ltd.) and Th1/Th2 ratio was derived from the following equation:

Th1/Th2=CD(+)CD8(−)/CD4F-CD(+)CD(−)

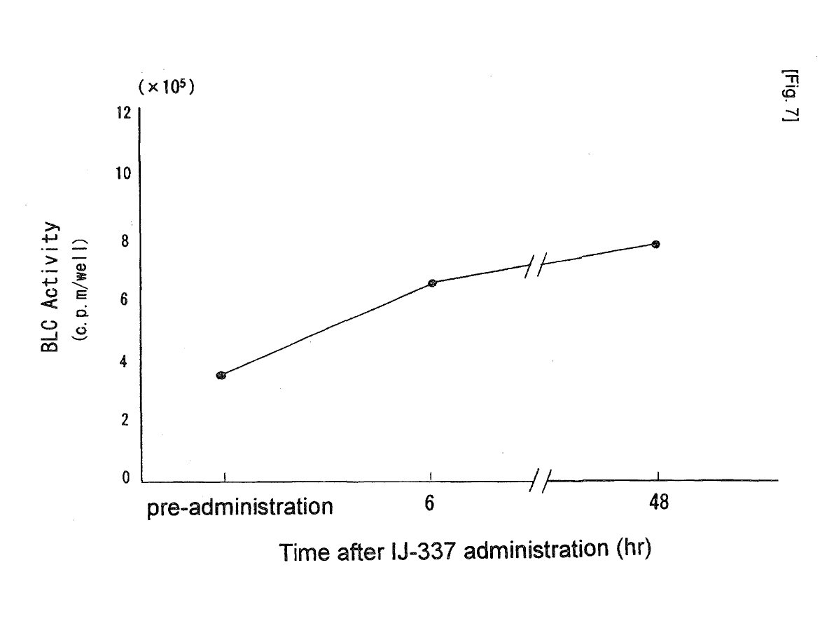

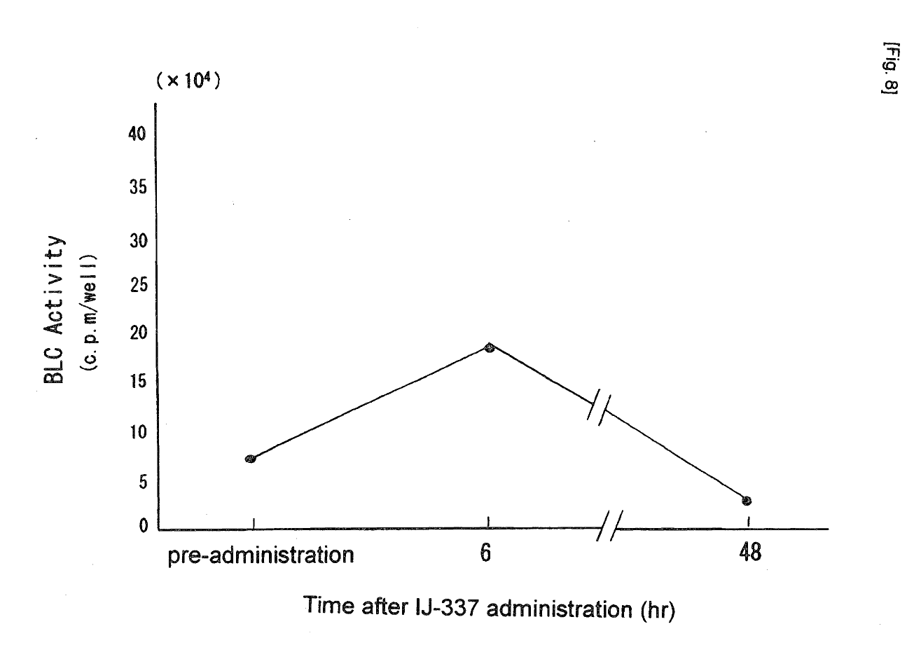

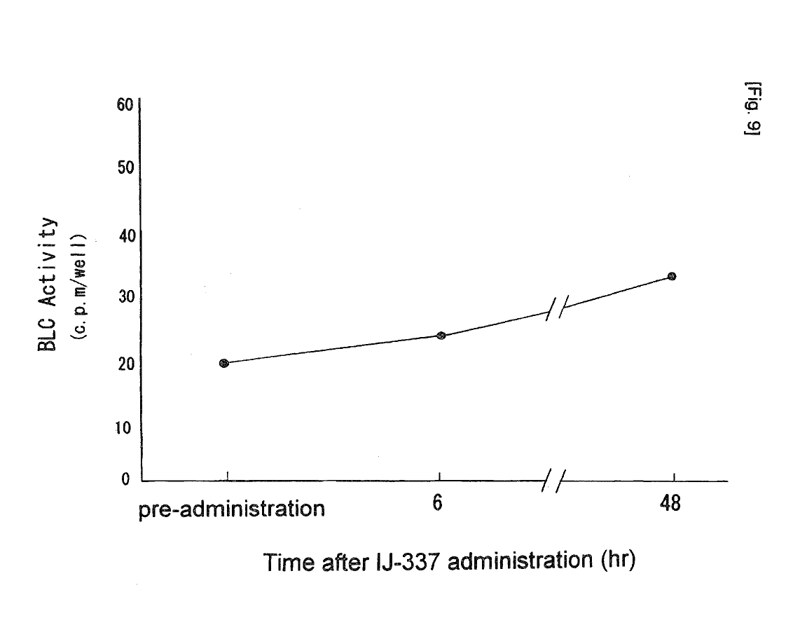

The enhancement effects of NK/NKT by IJ-337 are shown in FIG. 7 and macrophage (FIG. 8) activities by IJ-337 are shown in FIG. 8 and the relative enhancement effects of Th1/Th2 by IJ-337 are shown in FIG. 9. The NK/NKT cell activity indicated a onset value (6.4 cpm/well) 1.8-fold higher than the onset value (or before administration; 3.6 cpm/well) 6 hr after IJ-337 administration. NK/NKT cell activity continued to increase thereafter to register a value (7.9 cpm/well) 2.2-fold higher than the onset value 48 hr after IJ-337 administration (FIG. 7). Additionally, dogs treated with either INF-omega or INF-gamma in a similar manner indicated the NK/NKT cell activity to attain a level 2-fold higher when the results of these INF were compared with that of IJ-337 (data not shown).

The macrophage activity indicated a 2.5-fold (18.5 cpm/well) increase and recovered to basal levels compared to the pre-administration BCL active value (7.4 cpm/well) at 6 hr and 48 hr after IJ-337 administration, respectively (FIG. 8). Changes in the Th1/Th2 value were analogous to those of NK/NKT cell activity; the Th1/Th2 value indicated 1.2-fold (23.7) and 1.6-fold (31.9) increases compared with pre-administration value (20.4) at 6 and 48 hr after IJ-337 administration, respectively (FIG. 9).

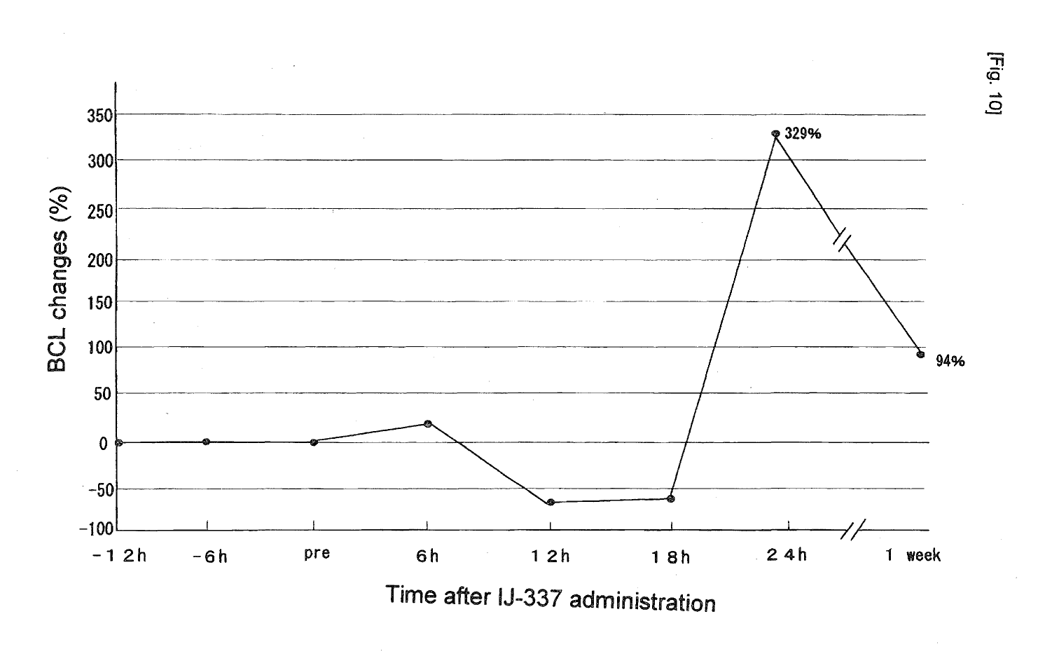

Furthermore, the NK/NKT cell activity was monitored more closely at 6-hr intervals and 1 week after IJ-337 administration in order to study more details on the changes in NK/NKT cell activity. Two other male dogs (10-11 kg) were accommodated and provided with feed pellets and drinking water ad libitum in a laboratory room maintained at a constant room temperature (25 degree Centigrade) and relative humidity of 50-60% for 1 week before experiment. IJ-337 (10 mg/kg) was orally administrated and observed for a 1-week period. For reference of the IJ-337 treatment effects, NK/NKT cell activities were monitored from 12 hr before administration and at 6-hr intervals thereafter; blood samples taken at 12 and 6 hr before IJ-337 administration via the jugular were collected in heparin-treated test-tubes (controls). The NK/NKT cell activity indicated the peak value (about 3.3-fold higher than the mean pre-administration values) 24 hr and showed a recovery tendency to the basal NK/NKT cell activity values 1 week after IJ-337 administration (FIG. 10).

Both NK/NKT cell and macrophage activities increased after IJ-337 administration, and the NK-NKT cell activity-related Th1/Th2 ratio increased analogously as well. Enhancement of B cell activity can thus be expected. As such, IJ-337 administration may be expected to serve as a potential immunotherapy for treating various immunity-related diseases, such as cancers, tumors, allergies, rheumatism, atopy, etc.

(Suppressive Effects on Nociceptive Responses)

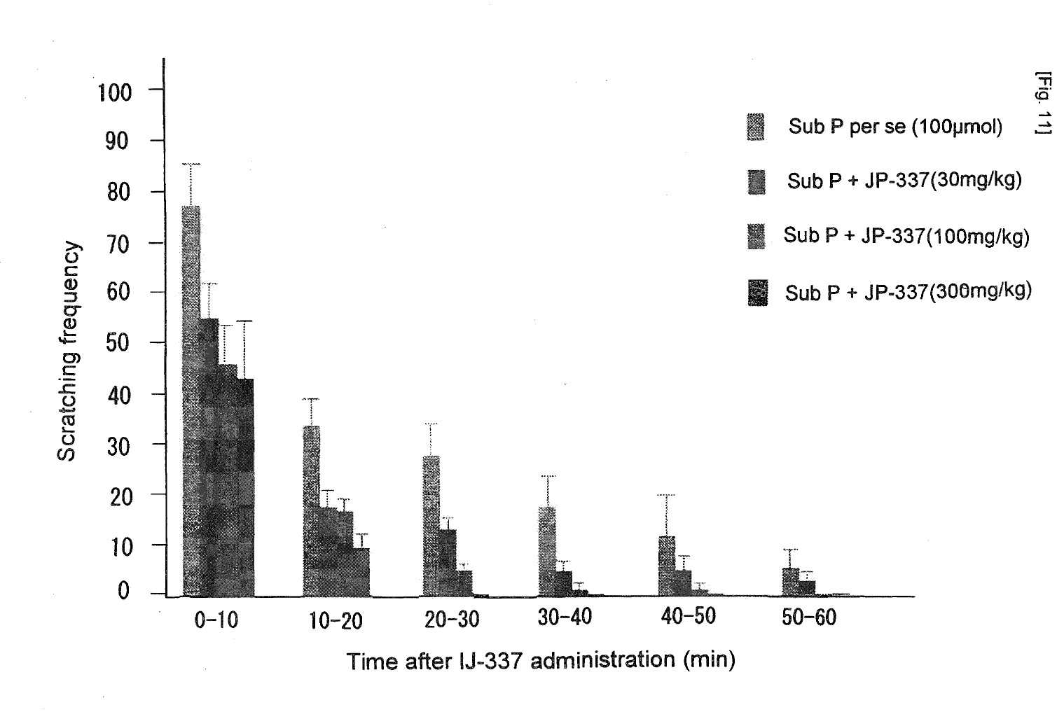

When IJ-337 was orally administered to male ddy mice (28-30 g), a time-related dose-dependent antinociceptive effect was established (FIG. 11). Injection of Substance P (SP) into the hindpaw planta of mice induces scratching responses in the treated mice. The antinociceptive effects of IJ-337 were therefore evaluated with SP-induced scratching as a nociceptive response in mice (evaluation using induced nociceptive stimulus screening test). Mice treated with SP per se (100 micro mol) were used a control group for comparison as Group 1. Groups 2, 3 and 4 were mice treated with 30, 100 and 300 mg/kg of IJ-337 30 min before SP injection to the hindpaw planta of mice. Eight animals were allocated for each group. Scratching responses were cumulatively counted at 10-min intervals and the IJ-337 effects were monitored for 60 min (10 min×6 observations) after SP injection. Anti-nociceptive effects appeared 20-30 min after administration, and the highest dose of 300 mg/kg indicated the most effective antinociceptive effects; the nociceptive agent (SP)-induced nociceptive responses was suppressed by IJ-337 in a dose-dependent manner (FIG. 11, Table 1). Based on these findings, IJ-337 may serve as a potential agent with buffering (anti-nociceptive) effects on chemically induced chronic pains and acute/chronic pains induced by injury/inflammation such as post-parturition and/or post-operative pain.

TABLE 1
Effects of IJ-337 against the nociceptive responses
induced by pain mediator Substance P (SP)

| Substance P (SP) | SP per se | IJ-337 pretreatment (mg/kg) | | |
|---|---|---|---|---|
| injection (min) | (100 micro mol) | SP + 30 | SP + 100 | SP + 300 |
| 0-10 | 78.2 ± 6.51 | 52.1 ± 8.41* | 47.5 ± 6.10* | 45.3 ± 8.50* |
| 10-20 | 32.5 ± 5.11 | 15.4 ± 3.51* | 15.2 ± 2.40* | 8.2 ± 2.40** |
| 20-30 | 27.1 ± 6.21 | 11.7 ± 1.21* | 4.3 ± 0.60** | 0.0 ± 0.00** |
| 30-40 | 18.5 ± 5.42 | 4.2 ± 1.31** | 1.2 ± 0.30** | 0.0 ± 0.00** |
| 40-50 | 12.6 ± 6.53 | 5.3 ± 2.11* | 0.5 ± 0.10** | 0.0 ± 0.00** |
| 50-60 | 5.2 ± 2.90 | 2.4 ± 0.52* | 0.0 ± 0.00** | 0.0 ± 0.00** |

When compared with responses induced with SP per se, IJ-337 significantly (*: p < 0.05; **: <0.01) suppressed the SP-induced nociceptive responses in a dose-dependent manner.

(Suppressive Effects on Neuropathic Pain)

Next, the effects of IJ-337 on sensory hyperalgesia due to nerve injury/damage were investigated using the aforementioned method by Bennett and Xie (Pain 1988; 33(1):87-107). Four-week-old male Wistar rats from a commercial source were acclimatized and accommodated with provision of feed and water ad libitum for 1 week. After the left sciatic nerve of the 5-week-old rats was surgically exposed and lightly ligated at 4 sites with absorbable sutures (post-operative day 0), means nociceptive responses were evaluated with time from postoperative day 7. As a reference, sham surgery was a performed on the right hindlimb of previously operated rats. Paw pressure, mechanical pressure as a nociceptive stimulus was applied on the both hindpaws of the rat to evaluate the nociceptive threshold. Four experimental animal groups were designated (6 rats/group). Group 1 were non-treated intact rats treated with physiological saline. Group 2 were intact rats treated i.p. with IJ-337 (30 mg/kg). Group 3 were sensory hyperalgesia rats (see the abovementioned reference) treated with physiological saline. Group 4 were sensory hyperalgesia rats by the Bennett method treated i.p. with 30 mg/kg IJ-337. A statistical tests on the nociceptive thresholds were operated on post-operative days 0, 3, 5, 7, 10, 14, 21 and 28. IJ-337 and physiological saline were given continuously after postoperative day. Note that administrations were given after testing the animals on experimental days.

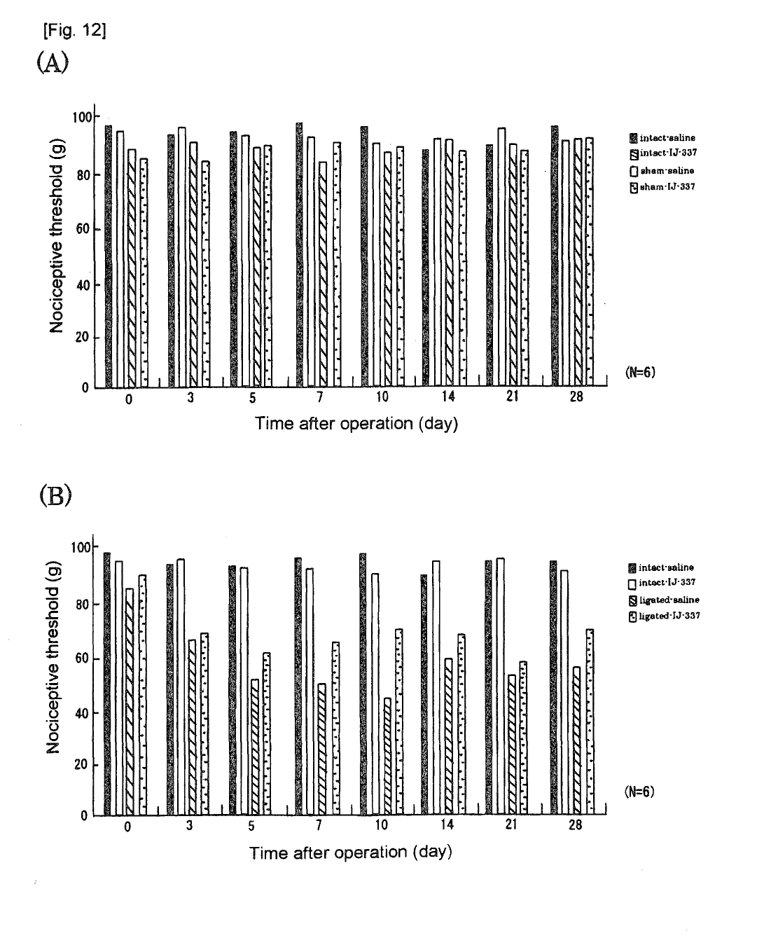

The results were shown in FIG. 12(A), 12(B), and Table 2. As shown in Table 2 no significant anti-nociceptive threshold changes in Groups 1 and 2, while Group 3 (sensory hyperalgesia rats by the Bennett method) indicated significant decreases in the nociceptive threshold compared with Group 1(p<0.05; shown a in Table 2). Furthermore, sensory hyperalgesia rats treated with IJ-337 (Group 4) indicated significant suppressions of the hyperalgesic effects (FIG. 12(B)). In all groups tested (Groups 1-4), the noceptive thresholds of the right intact hindpaws measured simultaneously with the same intensity did not show any remarkable changes (See FIG. 12(A)).

These findings advocate that IJ-337 elicits repair effects to relieve sensory hyperalgesia due to nerve damage. Additionally, when the nerve-repairing and nerve growth promoting effects are taken into consideration, IJ-337 may serve as a potentially useful agent for cerebral apoplexy-induced physical impairments and handicaps, peripheral neuropathy and symptoms like as irritations, etc.

TABLE 2
Buffering action of IJ-337 on hyperalgesia due to nerve injury/damage

| Postoperative | Group1 (g) | | Group2 (g) | | Group3 (g) | | Group4 (g) | |
|---|---|---|---|---|---|---|---|---|
| duration (day) | L | R | L | R | L | R | L | R |
| 0 | 96.7 ± 0.09 | 96.0 ± 0.09 | 93.7 ± 0.12 | 95.0 ± 0.06 | 83.3 ± 0.24^{a} | 88.7 ± 0.10 | 88.7 ± 0.31 | 85.0 ± 0.08 |
| 3 | 92.0 ± 0.11 | 93.0 ± 0.09 | 94.0 ± 0.09 | 95.7 ± 0.08 | 67.3 ± 0.18^{a} | 89.3 ± 0.08 | 68.3 ± 0.19 | 84.3 ± 0.10 |
| 5 | 94.0 ± 0.19 | 93.3 ± 0.10 | 91.0 ± 0.09 | 92.3 ± 0.13 | 54.7 ± 0.12^{a} | 87.7 ± 0.15 | 60.7 ± 0.10^{b} | 88.7 ± 0.17 |
| 7 | 94.7 ± 0.13 | 96.3 ± 0.09 | 89.7 ± 0.13 | 92.3 ± 0.08 | 51.0 ± 0.24^{a} | 85.0 ± 0.23 | 63.0 ± 0.14^{b} | 89.7 ± 0.17 |
| 10 | 94.7 ± 0.18 | 94.7 ± 0.07 | 87.7 ± 0.14 | 89.7 ± 0.19 | 44.0 ± 0.17^{a} | 87.0 ± 0.28 | 65.7 ± 0.17^{b} | 88.0 ± 0.13 |
| 14 | 86.7 ± 0.15 | 86.7 ± 0.19 | 91.7 ± 0.18 | 91.7 ± 0.21 | 57.0 ± 0.20^{a} | 91.3 ± 0.15 | 64.3 ± 0.26^{b} | 86.3 ± 0.09 |
| 21 | 90.3 ± 0.25 | 88.7 ± 0.25 | 91.7 ± 0.21 | 93.3 ± 0.08 | 48.0 ± 0.22^{a} | 89.0 ± 0.30 | 55.7 ± 0.32^{b} | 87.0 ± 0.13 |
| 28 | 89.0 ± 0.29 | 95.0 ± 0.28 | 85.7 ± 0.18 | 90.0 ± 0.14 | 50.3 ± 0.16^{a} | 93.3 ± 0.10 | 65.7 ± 0.30^{b} | 91.3 ± 0.10 |

Values are expressed as the mean ± S.E. Each group consisted of 6 rats. When Groups 3 vs 1 and Groups 4 vs 3 were compared, statistical significance of p < 0.05 (a) and <0.01 (b) were indicated respectively. In Groups 1 and 2, neither the left (L) nor right (R) hindlimb was operated. However, in Groups 3 and 4, the L hindlimb was operated by Bennet's Method while the R hindlimb was sham-operated.

(Improvement Effect of Diabetic Sensory Neuropathy)

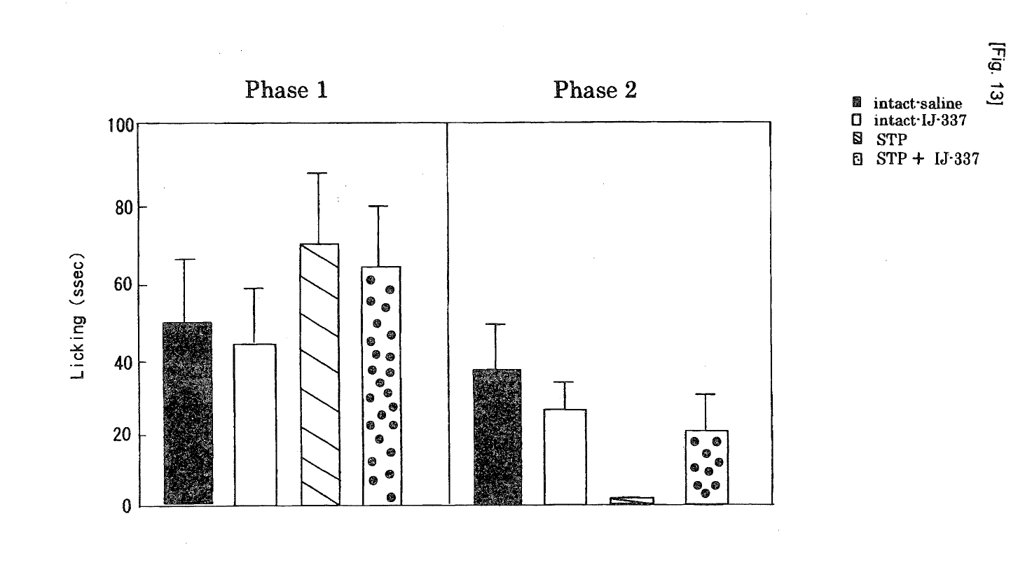

IJ-337 indicates the buffering action on streptozotocine (STZ)-induced diabetic sensory neuropathy in rats (FIG. 13). Commercially available 4-week-old male Wistar rats were acclimatized for 1 week with feed and water ad libitum. The 5-week-old rats were then treated with a single administration of 200 mg/kg of STZ via the tail vein. The blood glucose levels of STZ-treated rats were measured from the next day, and rats with blood sugar levels of 400 mg/dl and more were appropriated as diabetic rats. 30 mg/kg of IJ-337 was administrated intraperitoneally (i.p.) to these appropriated diabetic rats with 200 mg/kg of STZ simultaneously from STZ treatment day. On post-STZ treatment day 7, the nociceptive formalin test was performed. In the nociceptive formalin test, the nociceptive response is biphasic: i.e. the first phase nociceptive response (Phase 1; 10-20 min) and the second phase nociceptive response (Phase 2; 10-60 min) are shown. When 20 micro-liter of formaldehyde solution (1%) was injected into the handpaw planta of rat, the rat would lick the injected site (licking), if the stimulus was nociceptive. When IJ-337 was administered to rats, the licking duration was counted at 5-min intervals until 60 min after formaldehyde injection. The rats were divided into 4 groups. Groups 1 were non-treated intact rats (non-diabetic rats with physiological saline, Group 2 were non-treated intact rats with IJ-337, Group 3 were STZ-induced diabetic rats treated with physiological saline, Group 4 were STZ-induced diabetic rats treated with IJ-337. The results are indicated in FIG. 13.

The Phase 1 nociceptive responses in Groups 1 (49.4±15.2 sec) and 2 (44.6±13.6 sec) were not significantly different. The Phase 2 nociceptive responses in Group 1 (34.9±12.2 sec) and 2 (23.4±7.2 sec) indicated a normal trend without any significant difference; viz. a nociceptive response (licking time) shorter than Phase 1 responses.

As for Phase 1 nociceptive responses, licking duration at Phase 1 in Groups 3 (68.5±16.8 sec) were slightly extended compared with those of Groups 1 and 2. In Group 4 (61.1±16.3 sec) treated with IJ-337, the extension of Phase-1 licking duration was suppressed with a recovery tendency to levels close to those of Groups 1 and 2.

Phase-2 nociceptive responses, licking response in Group 3 (0.6±0.4 sec) of STZ-induced rats was further suppressed when compared with Groups 1 and 2 without almost licking response. Unlike Group 3, Phase-2 nociceptive responses durations indicated in Phase 2 in Group 4 (61.1±16.3 sec) treated with IJ-337 indicates a recovery tendency close to those of Groups 1 and 2.

IJ-337 (30 mg/kg, i.p.) administration significantly improved the peripheral neuropathy of STZ-induced diabetic rats (Group 4). When findings of brain neuron-repairing and nerve growth regeneration effects are taken into consideration, IJ-337 may serve as a potentially useful agent for cerebral apoplexy-induced physical impairments and handicaps, peripheral neuropathy and symptoms like as stress-induced irritations, etc.

(Antiulcerative Effects)

Oral administration of IJ-337 indicated anti-inflammatory and antiulcerative effects in Wistar rats.

Ulceration was induced in Wistar rats previously starved for 24 hr with ulcerogenic substance (US) such as acids (HCl), alkalis (NaOH), sodium chloride (NaCl), non-steroidal anti-inflammatory agents (indomethacin) and alcohols (ethanol). The rats were divided into 16-rats groups in each group. Eight of 16 rats in each group were orally given 1.0 mL each of HCl (0.6 M), NaOH (0.2 M), NaCl (25 w/v %), indomethacin (30 mg/kg) or ethanol (80 v/v %), while the remaining 8 rats in each group were orally administered with IJ-337 30 min before US treatment. US-treated rats were euthanized 8 hr after treatment and the whole stomach of each rat was surgically exposed to measure the ulcerative areas. IJ-337 was administered at 10, 100 and 200 mg/kg.

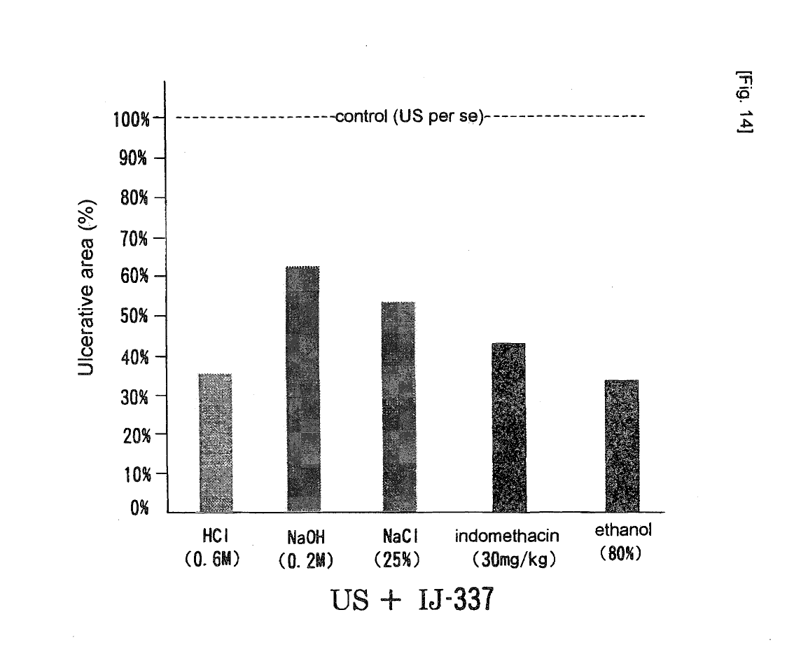

The antiulcerative effects of IJ-337 pretreatment were shown in FIG. 14. The figure indicates the ratio of the US-induced ulcerative area with pretreatment (200 mg/kg of IJ-337) to that without pretreatment. In rats without IJ-337 pretreatment, severe stomach ulceration was induced with frequent bleeding. The ulcerative areas (mm²) induced by HCl, NaOH, NaCl, indomethacin and ethanol registered 190.5±92.6, 164.2±42.6, 214.8±42.6, 90.8±14.6 and 143.0±28.5, respectively. However, in the group pretreated with the highest dose of 200 g/kg IJ-337, the respective ulcerative areas (mm²) were 69.0±42.9, 99.7±20.2, 112.8±82.2, 39.7±15.9 and 50.8±17.9. In short, when the ulcerative areas of the IJ-337 non-pretreated groups were expressed as 100%, those of the 200 mg/kg IJ-337-pretreated groups indicated 36.2%, 60.7%, 52.5%, 43.7% and 35.5%, respectively. Or, the suppressive effects of IJ-37 were 63.8%, 39.3%, 47.5%, 56.3% and 64.5%, respectively.

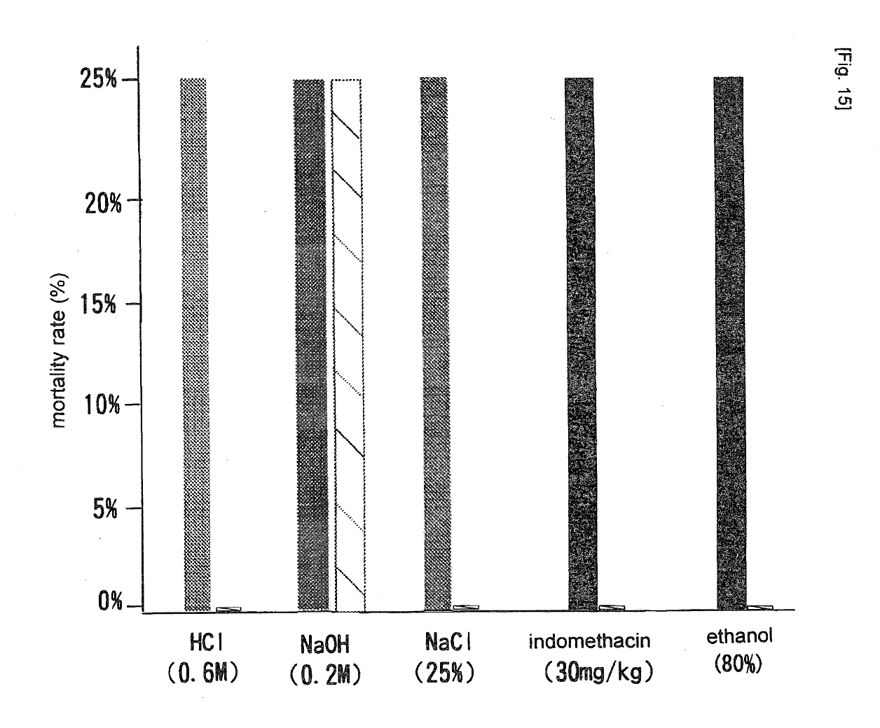

Additionally, the US-induced mortality cases in the HCl-, NaOH-, NaCl-, indomethacin- and ethanol-treated groups (without IJ-337 pretreatment) registered 2 in each groups. However, in groups pretreated with 200 mg/kg IJ-337, except for the NaOH-treated group ( 2/8 mortality case), none died in the other US-treated groups. The results are indicated in FIG. 15.

Based on the results, IJ-337 evidently elicited efficacious antiulcerative effects. As such, IJ-337 may serve as a potentially healing agent for stomach ulcers, chronic ulcer induced injuries, and injuries/inflammation associated with surgery and parturition, etc.

### INDUSTRIAL APPLICABILITY

The present invention furnishes novel neuron-repairing agenta, immunoenhancement agents, antidiabetics, analgesics, and antiulceratives.

1. An extract isolated from one of marine organisms including sea cucumbers, marine sponges and sea anemones, and marine organisms processed to remove internal organs, having the following characteristics:
(1) That the extract is almost colorless, slightly sticky liquid and has a slightly fishy odor, or an almost white powder having a slightly fishy odor, and when dissolved the extract in distilled water, an almost colorless solution is formed having a slight viscosity,
(2) That the water solution of the liquid or powder indicates maximum absorption at 264±10 nm.
(3) That the water solution of the liquid or powder indicates an acidity pH
(4) That the extract has at least one of the following effects: brain nerve cell repairs, nerve growth promotion, endogenous insulin release, immunosystem enhancement, antinociceptive effects, suppressing of neuropathic pain, improvement of diabetic sensory neuropathy, or anti-ulcerative effect.

2. An extract according to claim 1, wherein said extract is obtained by a step of:
boiling a liquid obtained from sea cucumbers processed to remove skin and internal organs thereof by liquefying under the sunlight, or extracting from sea cotton or sea anemones with boiled water;
subjecting a liquid obtained by boiling or the extracted liquid to a desalination;
treating the desalinated liquid with active carbon;
removing lipids and fats from the liquid treated with the active carbon; and
filtering the defatted liquid by a cationic ion-exchange resin and an anionic ion-exchange resin.

3. A nerve repair agent which contains effective amount of said extract described in claim 1.

4. An antidiabetic which contains effective amount of said extract described in claim 1.

5. An immunoenhancement agent which contains effective amount of said extract described in claim 1.

6. An analgesic which contains effective amount of said extract described in claim 1.

7. An anti-ulcerative or anti-inflammatory agent which contains effective amount of said extract described in claim 1.

8. A method of improving neuronal abnormalities in various species of organisms, inclusive of humans, animals and birds, with administrations of effective amount of said extract described in claim 1.

9. A method of improving endogenous insulin release in various species of organisms, inclusive of humans, animals and birds, with administrations of effective amount of said extract described in claim 1.

10. A method of enhancing immunosystems in various species of organisms, inclusive of humans, animals and birds, with administrations of effective amount of said extract described in claim 1.

11. A method of suppressing nociception in various species of organisms, inclusive of humans, animals and birds, with administrations of effective amount of said extract described in claim 1.

12. A method of suppressing neuronal impairment pain in various species of organisms, inclusive of humans, animals and birds, with administrations of effective amount of said extract described in claim 1.

13. A method of improving diabetic-derived sensory peripheral neuropathies in various species of organisms, inclusive of humans, animals and birds, with administrations of effective amount of said extract described in claim 1.

14. A method of ulcer treatment in various species of organisms, inclusive of humans, animals and birds, with administrations of effective amount of said extract described in claim 1.

15. A method of obtaining an extract isolated from one of marine organisms including sea cucumbers, marine sponges and sea anemones, or marine organisms processed to remove internal organs, which has at least one of the following effects: brain nerve cell repairs, nerve growth promotion, endogenous insulin release, immunosystem enhancement, antinociceptive effects, suppressing of neuropathic pain, improvement of diabetic sensory neuropathy, or anti-ulcerative effect, the method comprising:
a step of boiling a liquid obtained from sea cucumbers processed to remove skin and internal organs by liquefying under the sunlight or a step of extracting from cotton or sea anemones with boiled water,
a step of desalination of the liquid after boiling or the extract,
a step of treating the desalinated liquid with active carbon,
a step of removing lipids and fats from the liquid treated with active carbon and
a step of passing the defatted liquid through a cationic ion-exchange resin and an anionic ion-exchange resin.
